(19) United States
(12) Patent Application Publication
(10) Pub. No.: US 2024/0350226 A1
(43) Pub. Date: Oct. 24, 2024

(54) NON-CONTACT MONITORING OF NEONATE PHYSIOLOGICAL CHARACTERISTICS AND DEVELOPMENT

(71) Applicant: Covidien LP, Mansfield, MA (US)

(72) Inventors: Rakesh K. SETHI, Vancouver (CA); Paul S. ADDISON, Edinburgh (GB); Dean MONTGOMERY, Edinburgh (GB)

(21) Appl. No.: 18/640,342

(22) Filed: Apr. 19, 2024

Related U.S. Application Data
(60) Provisional application No. 63/460,551, filed on Apr. 19, 2023.

(51) Int. Cl. A61B 90/00; A61B 5/107; A61B 5/11

(52) U.S. Cl. CPC A61B 90/361; A61B 5/1079; A61B 5/1124; A61B 2503/04

(57) ABSTRACT

Use of non-contact monitoring systems to monitor a subject (e.g., neonate, baby, infant, child) to determine various physiological characteristics of the subject. Physical characteristic that can be monitored, measured, and/or determined include length, volume, weight, BMI, and head circumference. Other characteristics such as reactions to stimulus, development of motor skills, smiling, and even development of language can also be monitored.

### CROSS-REFERENCE TO RELATED APPLICATIONS

The present application claims priority to U.S. Provisional Patent Application No. 63/460,551, filed Apr. 19, 2023, and entitled “NON-CONTACT MONITORING OF NEONATE PHYSIOLOGICAL CHARACTERISTICS”, the entirety of which is hereby incorporated by reference.

### BACKGROUND

A number of standard measurements are undertaken for neonates in the neonatal intensive care unit (NICU). These measurements may include weight, length, size, BMI, head circumference, reactions to stimulus, etc., based on a linear time scale. As the child develops, additional monitoring may include development of motor skills and/or language.

These measurements and developments may be monitored by a clinician or caregiver, such as by taking physical measurements and/or observing motor skills during spot checks. Some of these measurements may be time consuming, and developments in, e.g., motor skills may not be on display during spot checks. Accordingly, there is a need for a better neonate monitoring system.

### SUMMARY

The present disclosure is directed to using non-contact monitoring systems to monitor a subject (e.g., neonate, baby, infant, child) and determine various physiological characteristics of the subject. Physical characteristic that can be monitored, measured, and/or determined include, but are not limited to, subject length, volume, weight, BMI, and head circumference. The length of various subject body parts or segments can also be measured, such the length of the subject's tibia, chest, or abdomen. Other characteristics such as reactions to stimulus, development of gross and/or fine motor skills, development of cognitive developmental skills, smiling, and even development of language can also be monitored using the non-contact monitoring system.

One particular embodiment described herein is a method of monitoring a subject with a non-contact monitoring system. The method includes detecting a first feature and a second feature of the subject in a field of view of a non-contact monitoring system using depth measurements, determining a measurement between the first feature and the second feature, and correlating the measurement to a length of a physiological characteristic of the subject.

Another particular embodiment described herein is a method of monitoring a subject. The method includes detecting a head of a subject in a field of view of a non-contact monitoring system, detecting a first feature and a second feature of the head of the subject with the non-contact monitoring system using depth measurements, determining a measurement between the first feature and the second feature, and correlating the measurement to a head circumference of the subject.

Yet another particular embodiment described herein is a method of monitoring a subject, the method including detecting a feature of the subject with a non-contact monitoring system using depth measurements, creating a three-dimensional map of the feature with the non-contact monitoring system from the depth measurements, and from the map, determining, with the non-contact monitoring system, a physiological characteristic of the subject.

The methods include monitoring the physiological characteristic over time and identifying a trend, results of which can be delivered to a clinician or caregiver, or saved for posterity.

Another particular embodiment described herein is a non-contact monitoring system that has a processor and a memory storing instructions that, when executed by the processor, cause the processor to determine a depth to a subject at a first feature and a second feature and determine a measurement between the first feature and the second feature indicative of a length of a physiological characteristic of the subject. In some embodiments, the measurement is indicative of a shape of a physiological characteristic.

Another particular embodiment described herein is a non-contact monitoring system that has a processor and a memory storing instructions that, when executed by the processor, cause the processor to detect a feature of the subject and create a three-dimensional map of the feature from depth measurements, and from the map, determine a physiological characteristic of the subject.

Other embodiments are also described and recited herein.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

These and other aspects of the technology described herein will be apparent after consideration of the Detailed Description and Drawing herein. It is to be understood, however, that the scope of the claimed subject matter shall be determined by the claims as issued and not by whether given subject matter addresses any or all issues noted in the Background or includes any features or aspects recited in the Summary.

### BRIEF DESCRIPTION OF THE DRAWING

FIG. 1 is a schematic diagram of an example non-contact monitoring system configured in accordance with various embodiments described herein, the non-contact monitoring system being positioned in relation to a crib.

FIG. 2 is a schematic diagram of another example non-contact monitoring system configured in accordance with various embodiments described herein.

FIG. 3 is a block diagram of a non-contact monitoring system configured in accordance with various embodiments described herein, the non-contact monitoring system including a computing device, a server, and an image capture device.

FIG. 4 is a perspective view of another example non-contact monitoring system configured in accordance with various embodiments described herein.

FIG. 5 is a perspective view of yet another non-contact monitoring system configured in accordance with various embodiments described herein, the non-contact monitoring system being positioned in relation to a bed.

FIG. 6 is a side view of yet another non-contact monitoring system configured in accordance with various embodiments described herein, the non-contact monitoring system being positioned in relation to a bed.

FIG. 7 is a step-wise method for monitoring a subject to determine length configured in accordance with various embodiments described herein.

FIG. 8 is a step-wise method for monitoring a subject to determine weight configured in accordance with various embodiments described herein.

FIG. 9 is a step-wise method for monitoring a subject to determine head circumference configured in accordance with various embodiments described herein.

FIG. 10 is another step-wise method for monitoring a subject to determine head circumference configured in accordance with various embodiments described herein.

### DETAILED DESCRIPTION

As described above, the present disclosure is directed to monitoring a subject (e.g., neonate, baby, infant, toddler) to determine various physical or physiological measurements or characteristics of the subject, such as weight, length, size, BMI, and head circumference, and/or to monitor subject development, such as reactions to stimulus, development of motor skills, and development of language.

The monitoring of the subject may be accomplished with a non-contact monitoring system that uses a video signal of the subject and identifying physiologically relevant areas within the video image (such as the subject's head, face, neck, arms, legs, or torso). The non-contact monitoring system may then determine a distance between identified physiologically relevant areas and use this measurement to calculate various physical or physiological measurements of the subject. In some embodiments, the calculation is based on known or established correlations between the measured distance and the physical or physiological measurements of the subject, and therefore the calculation may be an estimate of the physical or physiological measurements of the subject. In other embodiments, depth measurements and/or distance measurements are processed by artificial intelligence to calculate estimates on various physical or physiological characteristics of the subject.

In some embodiments, the physiologically relevant areas may be identified using depth measurements acquired by the non-contact monitoring system. In some embodiments, the non-contact monitoring system may use vision-based artificial intelligence (AI) methods to identify the relevant areas or features. In some embodiments, both depth measurements and AI methods are used to identify relevant areas. The system can be used in a medical or commercial setting, such as a NICU in a hospital, or in a residential setting.

With the non-contact monitoring systems, signals representative of the topography, and optionally movement, of the subject are detected by a camera or camera system that views but does not contact the subject. The camera or camera system may utilize any or all of depth signals, color signals (e.g., RGB signals), and IR signals. With appropriate selection and filtering of the signals detected by the camera, the physiologic contribution by each of the detected signals can be isolated and measured.

The monitoring methods described herein may be done over time, e.g., days, weeks, months, etc., to determine progression of the monitored parameters. The methods are particularly useful for alerting caregivers (e.g., clinician, parent, etc.) of a subject's development, providing a record thereof over time, and in some cases, alerting the caregiver to potential concerns regarding a subject's development or lack thereof.

In some embodiments, the non-contact monitoring system used for the non-contact monitoring of the subject is developed to identify features of the subject and obtain measurements (e.g., linear measurements, shaped-based measurements, etc.) of or between the features, which measurements can then be used to correlate or extrapolate the measured features to relevant parameters and, in some cases, monitor the change in those parameters over time. In some embodiments, the non-contact monitoring system can utilize AI to identify the features and/or correlate or extrapolate the data to the desired parameter.

In some embodiments, the non-contact system receives a video signal of the subject and from the video signal extracts a distance or depth signal to the relevant area. Multiple depth signals extracted from the video signal can be used to provide a topographical map of some or all of the subject. Alternatively or in conjunction with using a video signal to extract depth signals, the system can also receive a light intensity signal reflected from the subject, and from the reflected light intensity signal calculate a depth or distance. In some embodiments, the light intensity signal is a reflection of a pattern or feature (e.g., using visible color or infrared) projected onto the subject, such as by a projector.

In addition to determining depth or distance measurements, the systems and methods described herein may also use obtained depth or distance information to determine movement or motion or a subject.

The depth sensing feature of the system provides a measurement of the distance or depth between the detection system and one or more points on the subject. One or more video cameras may be used to determine the depth, and change in depth, from the system to the subject. For example, when two or more cameras, set at a fixed distance apart, are used, they offer stereo vision due to the slightly different perspectives of the scene from which distance information is extracted. When distinct features are present in the scene, the stereo image algorithm can find the locations of the same features in the multiple image streams. However, if an object is featureless (e.g., a smooth surface with a monochromatic color), then the depth camera system may have difficulty resolving the perspective differences. By including an image projector to project features (e.g., in the form of dots, pixels, etc., visual or IR) onto the scene, this projected feature can be monitored over time to produce an estimate of location and any change in location of an object.

In the following description, reference is made to the accompanying drawing that forms a part hereof and in which is shown by way of illustration at least one specific embodiment. The following description provides additional specific embodiments. It is to be understood that other embodiments are contemplated and may be made without departing from the scope or spirit of the present disclosure. The following detailed description, therefore, is not to be taken in a limiting sense. While the present disclosure is not so limited, an appreciation of various aspects of the disclosure will be gained through a discussion of the examples, including the figures, provided below. In some instances, a reference numeral may have an associated sub-label consisting of a lower-case letter to denote one of multiple similar components. When reference is made to a reference numeral without specification of a sub-label, the reference is intended to refer to all such multiple similar components.

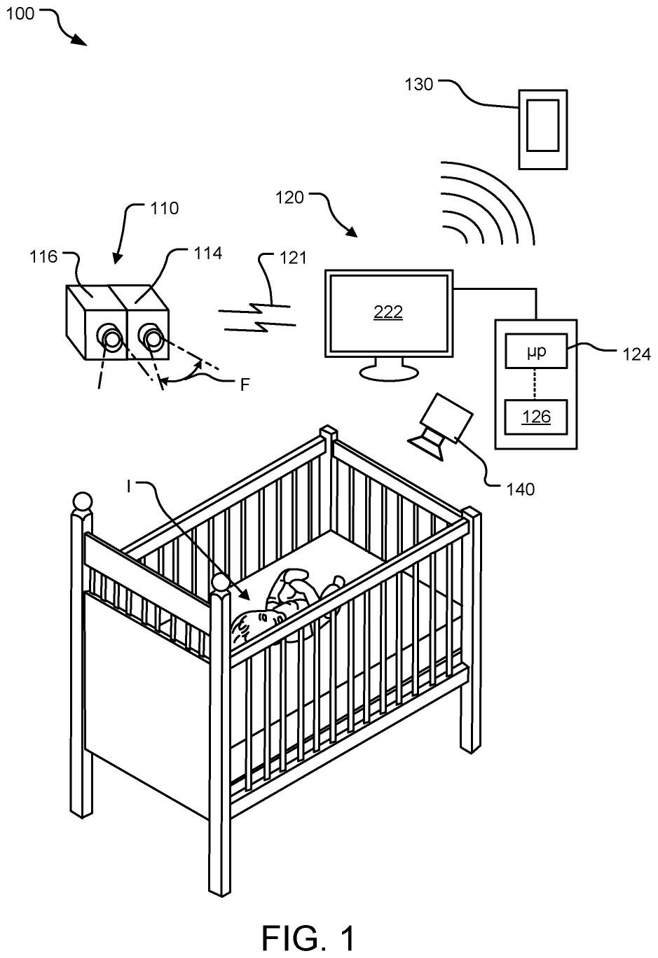

FIG. 1 shows a non-contact subject monitoring system 100 and a subject I, which, in this particular non-limiting example, is an infant in a crib. It is noted that the systems and methods described herein are not limited to a crib, but may be used with a bassinette, an incubator, an isolette, a bed, or any other place where the subject is. Similarly, the systems and methods described herein are not limited to monitoring an infant, and can be used for monitoring a subject of any age.

The system 100 includes a non-contact detector system 110 placed remote from the subject I. In this embodiment, the detector system 110 includes a camera system 114, particularly, a camera that includes an infrared (IR) detection feature. The camera system 114 may be a depth sensing camera system, such as a Kinect camera from Microsoft Corp. (Redmond, Washington) or a RealSense™ D415, D435 or D455 camera from Intel Corp. (Santa Clara, California).

The camera system 114 may operate at a set frame rate, which is the number of image frames taken per second (or other time period). Example frame rates include 20, 30, 40, 50, or 60 frames per second, greater than 60 frames per second, or other values between those. Frame rates of 20-30 frames per second produce useful signals, though frame rates above 100 or 120 frames per second are helpful in avoiding aliasing with light flicker (for artificial lights having frequencies around 50 or 60 Hz).

The camera system 114 is remote from the subject I, in that it is spaced apart from and does not physically contact the subject I. The camera system 114 may be positioned in close proximity to or on the crib. The camera system 114 has a field of view F that encompasses at least a portion of the subject I.

The field of view F is selected to be at least the upper torso of the subject. However, as it is common for young children and infants to move within the confines of their crib, bed or other sleeping area, the entire area potentially occupied by the subject I (e.g., the crib) may be the field of view F.

The camera system 114 includes a depth sensing camera that can detect a distance between the camera system 114 and objects in its field of view F. Such information can be used to determine that the subject is within the field of view of the camera system 114 and determine a region of interest (ROI) to monitor on the subject. The ROI may be the entire field of view F or may be less than the entire field of view F. Once an ROI is identified, the distance to the desired feature is determined and the desired measurement(s) can be made.

The ROI is monitored over time. The distance from the ROI on the subject I to the camera system 114 is measured by the system 100. Generally, the camera system 114 detects a distance between the camera system 114 and the surface within the ROI. With this distance, the system 100 can determine the presence of the subject I and with multiple points, can identify the location on the subject (e.g., the head). From multiple points, various parameters (e.g., circumference of the head) can be calculated. Any change in the parameter measurement or calculation over time (e.g., days, weeks, months, etc.) is acknowledged. However, a change in depth of points in a short time period (e.g., seconds, minutes) can represent movements of the subject I.

In some embodiments, the system 100 determines a skeleton outline of the subject I to identify a point or points from which to extrapolate the ROI. For example, a skeleton may be used to find a center point of a chest, shoulder points, waist points, hands, head, and/or any other points on a body. These points can be used to determine the ROI. In other embodiments, instead of using a skeleton, other points are used to establish an ROI. For example, a face may be recognized, and a torso and waist area inferred in proportion and spatial relation to the face.

In another example, the subject I may wear a specially configured piece of clothing that identifies points on the body such as the torso or the arms. The system 100 may identify those points by identifying the indicating feature of the clothing. Such identifying features could be a visually encoded message (e.g., bar code, QR code, etc.), or a brightly colored shape that contrasts with the rest of the subject's clothing, etc. In some embodiments, a piece of clothing worn by the subject may have a grid or other identifiable pattern on it to aid in recognition of the subject and/or their movement. In some embodiments, the identifying feature may be stuck on the clothing using a fastening mechanism such as adhesive, a pin, etc., or stuck directly on the subject's skin, such as by adhesive. For example, a small sticker or other indicator may be placed on a subject's hands that can be easily identified from an image captured by a camera.

In some embodiments, the system 100 may receive a user input to identify a starting point for defining an ROI. For example, an image may be reproduced on an interface, allowing a user of the interface to select a point on the subject from which the ROI can be determined (such as a point on the head). Other methods for identifying a subject, points on the subject, and defining an ROI may also be used.

However, if the ROI is essentially featureless (e.g., a smooth surface with a monochromatic color, such as a blanket or sheet covering the subject I), then the camera system 114 may have difficulty resolving the perspective differences. To address this, the system 100 can include a projector 116 to project individual features (e.g., dots, crosses or Xs, lines, individual pixels, etc.) onto objects in the ROI; the features may be visible light, UV light, infrared (IR) light, etc. The projector may be part of the detector system 110 or the overall system 100.

The projector 116 generates a sequence of features over time on the ROI from which is monitored and measured the reflected light intensity. A measure of the amount, color, or brightness of light within all or a portion of the reflected feature over time is referred to as a light intensity signal. The camera system 114 detects the features from which this light intensity signal is determined. In an embodiment, each visible image projected by the projector 116 includes a two-dimensional array or grid of pixels, and each pixel may include three color components—for example, red, green, and blue (RGB). A measure of one or more color components of one or more pixels over time is referred to as a “pixel signal,” which is a type of light intensity signal. In another embodiment, when the projector 116 projects an IR feature, which is not visible to a human eye, the camera system 114 includes an infrared (IR) sensing feature. In another embodiment, the projector 116 projects a UV feature. In yet other embodiments, other modalities including millimeter-wave, hyper-spectral, etc., may be used.

The projector 116 may alternately or additionally project a featureless intensity pattern (e.g., a homogeneous, a gradient or any other pattern that does not necessarily have distinct features, or a pattern of random intensities). In some embodiments, the projector 116, or more than one projector, can project a combination of a feature-rich pattern and featureless patterns on to the ROI.

The light intensity of the image reflected by the subject surface is detected by the detector system 110.

The measurements (e.g., one or more of depth signal, RGB reflection, light intensity) are sent to a computing device 120 through a wired or wireless connection 121. The computing device 120 includes a display 122, a processor 124, and hardware memory 126 for storing software and computer instructions. Sequential image frames of the subject I are recorded by the video camera system 114 and sent to the computing device 120 for analysis by the processor 124. The display 122 may be remote from the computing device 120, such as a video screen positioned separately from the processor and memory. Other embodiments of the computing device 120 may have different, fewer, or additional components than shown in FIG. 1. In some embodiments, the computing device may be a server. In other embodiments, the computing device of FIG. 1 may be connected to a server. The captured images (e.g., still images or video) can be processed or analyzed at the computing device and/or at the server to create a topographical map or image to identify the subject I and any other objects with the ROI.

In some embodiments, the computing device 120 is operably connected (e.g., wirelessly, via WiFi connectivity, cellular signal, Bluetooth™ connectivity, etc.) to a remote device 130 such as a smart phone, tablet, or merely a screen. The remote device 130 can be remote from the computing device 120 and the subject I, for example, in an adjacent or nearby room. The computing device 120 may send a video feed to the remote device 130, showing e.g., the subject I and/or the field of view F. Additionally or alternately, the computing device 120 may send instructions to the remote device 130 to advise a clinician or caregiver of the status of the subject I.

Also in some embodiments, the computing device 120 is operably connected to a microphone 140 for receiving audio. The microphone 140 can be integrated into the computer device 120 or into the detector system 110, or may be a stand-alone device.

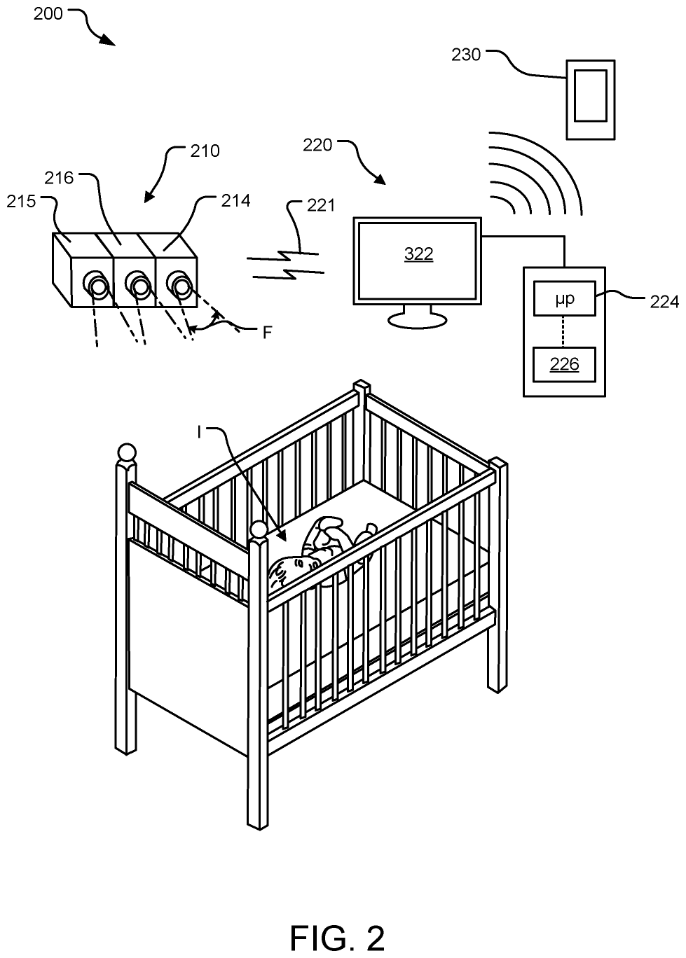

FIG. 2 shows another non-contact subject monitoring system 200 and a subject I, in this example, an infant in a crib. The system 200 includes a non-contact detector 210 placed remote from the subject I. In this embodiment, the detector 210 includes a first camera 214 and a second camera 215, at least one of which includes an infrared (IR) camera feature. The cameras 214, 215 are positioned so that their ROIs at least intersect, in some embodiments, completely overlap. The detector 210 also includes an IR projector 216, which projects individual features (e.g., dots, crosses or Xs, lines, or a featureless pattern, or a combination thereof etc.) onto the subject I in the ROI. The projector 216 can be separate from the detector 210 or integral with the detector 210, as shown in FIG. 2. In some embodiments, more than one projector 216 can be used. Both cameras 214, 215 are aimed to have features projected by the projector 216 to be in their ROI. The cameras 214, 215 and the projector 216 are remote from the subject I, in that they are spaced apart from and do not contact the subject I. In this implementation, the projector 216 is physically positioned between the cameras 214, 215, whereas in other embodiments it may not be so.

The distance from the ROI to the cameras 214, 215 is measured by the system 200. Generally, the cameras 214, 215 detect a distance between the cameras 214, 215 and the projected features on a surface within the ROI. The light from the projector 216 hitting the surface is scattered/diffused in all directions; the diffusion pattern depends on the reflective and scattering properties of the surface. The cameras 214, 215 also detect the light intensity of the projected individual features in their ROIs. From the distance and the light intensity, the presence of the subject I and any objects are monitored, as well as any movement of the subject I or objects.

The detected images, diffusion measurements and/or reflection pattern are sent to a computing device 220 through a wired or wireless connection 221. The computing device 220 includes a display 222, a processor 224, and hardware memory 226 for storing software and computer instructions. The display 222 may be remote from the computing device 220, such as a video screen positioned separately from the processor and memory. In other embodiments, the computing device of FIG. 2 may be connected to a server. The captured images (e.g., still images or video) can be processed or analyzed at the computing device and/or at the server to create a topographical map or image to identify the subject I and any other objects with the ROI.

In some embodiments, the computing device 220 is operably connected (e.g., wirelessly, via WiFi connectivity, cellular signal, Bluetooth™ connectivity, etc.) to a remote device 230 such as a smart phone, tablet, or merely a screen. The remote device 230 can be remote from the computing device 220 and the subject I, for example, in an adjacent or nearby room. The computing device 220 may send a video feed to the remote device 230, showing, e.g., the subject I and/or the field of view F.

The computing device 120, 220 has an appropriate memory, processor, and software or other program to evaluate the ROI image, identify features of the subject, and maintain a database. The computing device 120, 220 can be trained with vision-based artificial intelligence (AI) methods to learn to identify particular physical features of the subject, including the face and/or head of the subject, feet of the subject, and the position of the subject. The computing device 120, 220 can be trained using any standard AI model and standard methods, e.g., utilizing numerous data points to create a dataset of images.

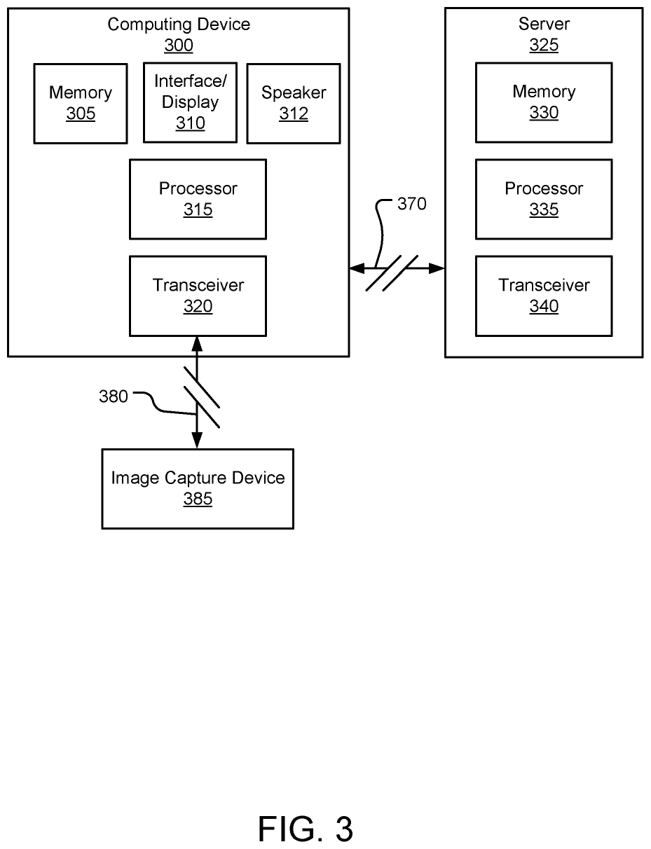

FIG. 3 is a block diagram illustrating a system including a computing device 300, a server 325, and an image capture device 385 (e.g., a camera, e.g., the camera system 114 or cameras 214, 215). In various embodiments, fewer, additional and/or different components may be used in the system.

The computing device 300 includes a processor 315 that is coupled to a memory 305. The processor 315 can store and recall data and applications in the memory 305, including applications that process information and send commands/signals according to any of the methods disclosed herein. The processor 315 may also display objects, applications, data, etc. on an interface/display 310 and/or provide an audible alert via a speaker 312. The processor 315 may also or alternately receive inputs through the interface/display 310. The processor 315 is also coupled to a transceiver 320. With this configuration, the processor 315, and subsequently the computing device 300, can communicate with other devices, such as the server 325 through a connection 370 and the image capture device 385 through a connection 380. For example, the computing device 300 may send to the server 325 information determined about a subject from images captured by the image capture device 385, such as depth information of a subject or object in an image.

The server 325 also includes a processor 335 that is coupled to a memory 330 and to a transceiver 340. The processor 335 can store and recall data and applications in the memory 330. With this configuration, the processor 335, and subsequently the server 325, can communicate with other devices, such as the computing device 300 through the connection 370.

The computing device 300 may be, e.g., the computing device 120 of FIG. 1 or the computing device 220 of FIG. 2. Accordingly, the computing device 300 may be located remotely from the image capture device 385, or it may be local and close to the image capture device 385 (e.g., in the same room). The processor 315 of the computing device 300 may perform any or all of the various steps disclosed herein. In other embodiments, the steps may be performed on a processor 335 of the server 325. In some embodiments, the various steps and methods disclosed herein may be performed by both of the processors 315 and 335. In some embodiments, certain steps may be performed by the processor 315 while others are performed by the processor 335. In some embodiments, information determined by the processor 315 may be sent to the server 325 for storage and/or further processing.

The devices shown in the illustrative embodiment may be utilized in various ways. For example, either or both of the connections 370, 380 may be varied. For example, either or both the connections 370, 380 may be a hard-wired connection. A hard-wired connection may involve connecting the devices through a USB (universal serial bus) port, serial port, parallel port, or other type of wired connection to facilitate the transfer of data and information between a processor of a device and a second processor of a second device. In another example, one or both of the connections 370, 380 may be a dock where one device may plug into another device. As another example, one or both of the connections 370, 380 may be a wireless connection. These connections may be any sort of wireless connection, including, but not limited to, Bluetooth connectivity, Wi-Fi connectivity, infrared, visible light, radio frequency (RF) signals, or other wireless protocols/methods. For example, other possible modes of wireless communication may include near-field communications, such as passive radio-frequency identification (RFID) and active RFID technologies. RFID and similar near-field communications may allow the various devices to communicate in short range when they are placed proximate to one another. In yet another example, the various devices may connect through an internet (or other network) connection. That is, one or both of the connections 370, 380 may represent several different computing devices and network components that allow the various devices to communicate through the internet, either through a hard-wired or wireless connection. One or both of the connections 470, 480 may also be a combination of several modes of connection.

The configuration of the devices in FIG. 3 is merely one physical system on which the disclosed embodiments may be executed. Other configurations of the devices shown may exist to practice the disclosed embodiments. Further, configurations of additional or fewer devices than the ones shown in FIG. 3 may exist to practice the disclosed embodiments. Additionally, the devices shown in FIG. 3 may be combined to allow for fewer devices than shown or separated such that more than the three devices exist in a system. It will be appreciated that many various combinations of computing devices may execute the methods and systems disclosed herein. Examples of such computing devices may include other types of infrared cameras/detectors, night vision cameras/detectors, other types of cameras, radio frequency transmitters/receivers, smart phones, personal computers, servers, laptop computers, tablets, RFID enabled devices, or any combinations of such devices.

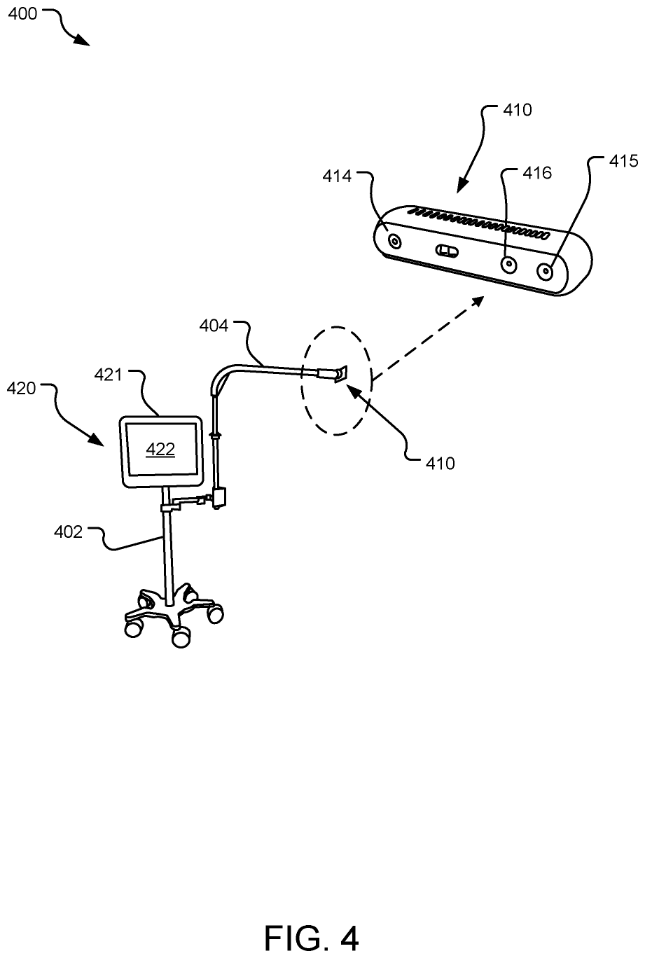

Alternate configurations of non-contact monitoring systems are shown in FIGS. 4, 5 and 6.

FIG. 4 shows a portable non-contact subject monitoring system 400 that includes a non-contact detector 410 and a computing device 420. In this embodiment, the non-contact detector 410 and the computing device 420 are generally fixed in relation to each other and the system 400 is readily moveable in relation to the subject to be monitored. The detector 410 and the computing device 420 are supported on a trolley or stand 402, with the detector 410 on an arm 404 that is pivotable in relation to the stand 402 as well as adjustable in height. The system 400 can be readily moved and positioned where desired.

The detector 410 includes a first camera 414 and a second camera 415, at least one of which includes an infrared (IR) camera feature. The detector 410 also includes an IR projector 416, which projects individual features (e.g., dots, crosses or Xs, lines, or a featureless pattern, or a combination thereof etc.).

The detector 410 may be wired or wireless connected to the computing device 420. The computing device 420 includes a housing 421 with a touch screen display 422, a processor (not seen), and hardware memory (not seen) for storing software and computer instructions.

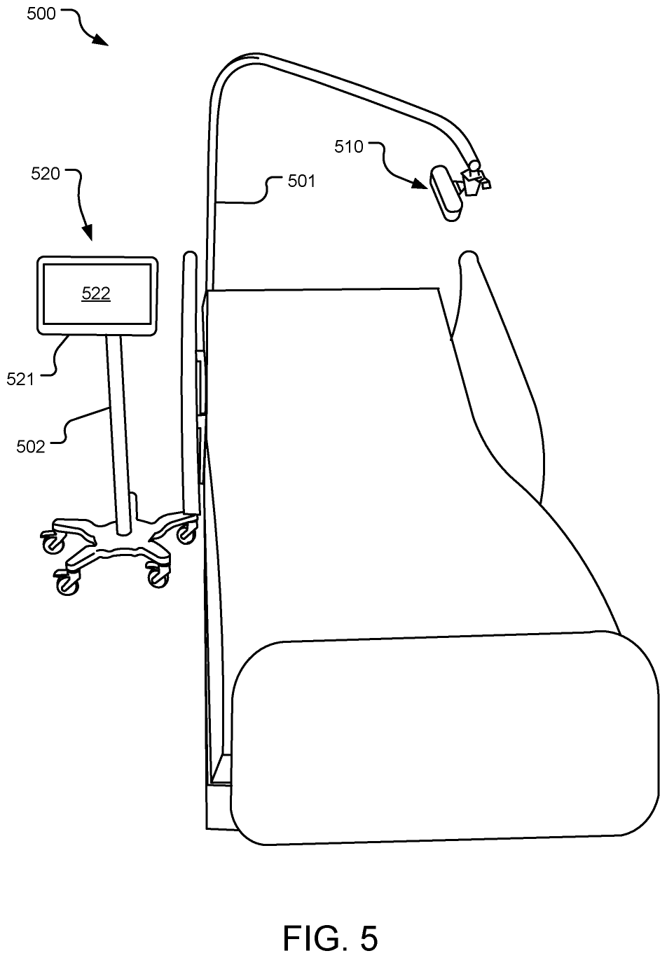

FIG. 5 shows a semi-portable non-contact subject monitoring system 500 that includes a non-contact detector 510 and a computing device 520. In this embodiment, the non-contact detector 510 is in a fixed relation to the subject to be monitored and the computing device 520 is readily moveable in relation to the subject.

The detector 510 is supported on an arm 501 that is attached to a bed, in this embodiment, a hospital bed, although the detector 510 and the arm 501 can be attached to a crib, a bassinette, an incubator, an isolette, or other bed-type structure. In some embodiments, the arm 501 is pivotable in relation to the bed as well as adjustable in height to provide for proper positioning of the detector 510 in relation to the subject.

The detector 510 may be wired or wireless connected to the computing device 520, which is supported on a moveable trolley or stand 502. The computing device 520 includes a housing 521 with a touch screen display 522, a processor (not seen), and hardware memory (not seen) for storing software and computer instructions.

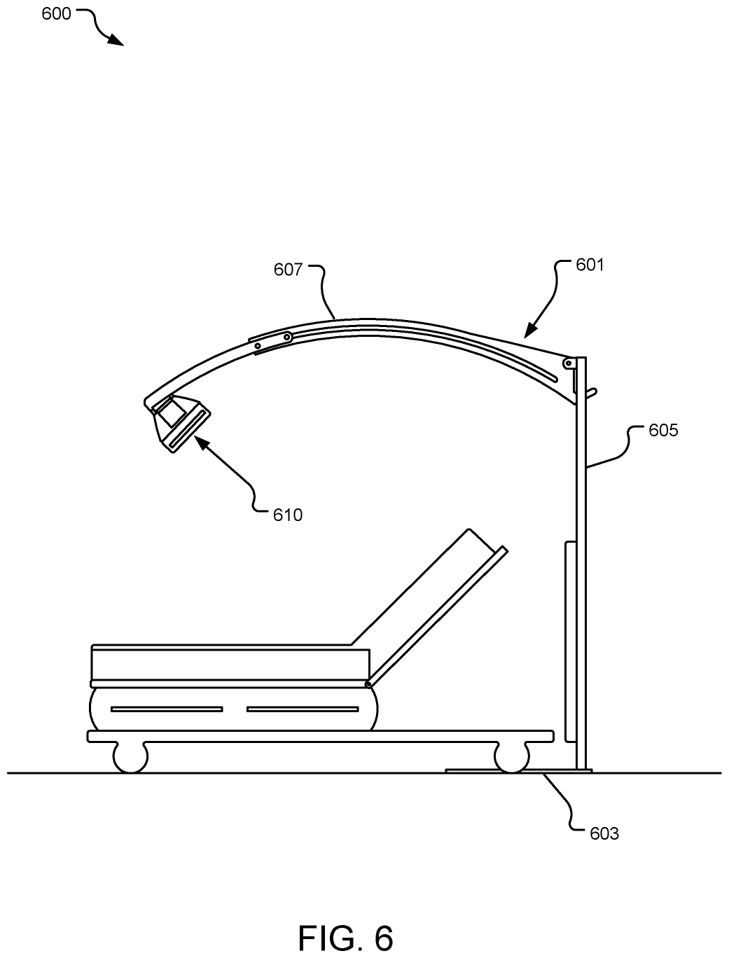

FIG. 6 shows a non-portable non-contact subject monitoring system 600 that includes a non-contact detector 610 and a computing device (not seen in FIG. 6). In this embodiment, at least the non-contact detector 610 is generally fixed in a location, configured to have the subject to be monitored moved into the appropriate position to be monitored.

The detector 610 is supported on a stand 601 that is free standing, the stand having a base 603, a frame 605, and a gantry 607. The gantry 607 may have an adjustable height, e.g., movable vertically along the frame 605, and may be pivotable, extendible and/or retractable in relation to the frame 605. The stand 601 is shaped and sized to allow a bed or bed-type structure to be moved (e.g., rolled) under the detector 610.

The non-contact monitoring systems and methods of this disclosure utilize depth (distance) information between the camera(s) and a subject to identify features of the subject and obtain measurements of the features or between the features, to correlate or extrapolate the measured features to relevant parameters, and monitor the change in those parameters over time. The measurements of the features may be linear measurements (single or multiple linear measurements), shape measurements, or other measurements of distance between multiple points. The parameter may be, e.g., subject length, size, weight, BMI, head circumference, the length of a specific body part or segment of the subject (e.g., length of subject chest, abdomen, tibia, etc.), reactions to stimulus, development of fine and/or gross motor skills, and even development of language and other cognitive developments.

In some embodiments, methods of using the previously described non-contact monitoring system may generally include using the non-contact monitoring system to identify at least a first feature on a subject and a second feature on a subject and determining a distance between the first feature and the second feature. Determining the distance between a first and second feature may include determining the depth distance between a camera and the first feature and a depth distance between a camera and the second feature, and then using these measurement to determine an orthogonal measurement between the two features. Depending on the first and second feature, the determined distance can then be used to directly or indirectly determine a physical or physiological characteristic of the subject.

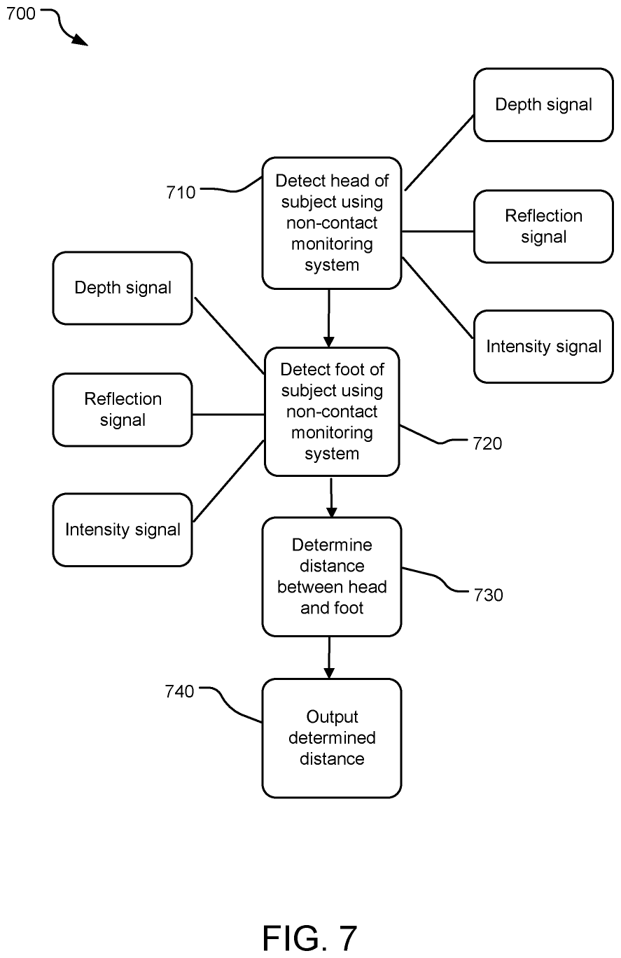

In some embodiments, the measured distance will be equal to the physical or physiological characteristic, and in such embodiments, no further calculations or correlations are required in order to obtain the physical or physiological characteristic. For example, and as discussed in further detail below with respect to FIG. 7, the desired physical or physiological characteristic may be subject length or height. In such embodiments, the first feature may be the top of the subject's head and the second feature may be the bottom of the subject's foot, in which case the distance between the first feature and the second feature is the subject's length or height when the subject is positioned generally in a straight line. In such embodiments, no further calculations or correlations may be required, as the measured distance is equal to the subject's length or height.

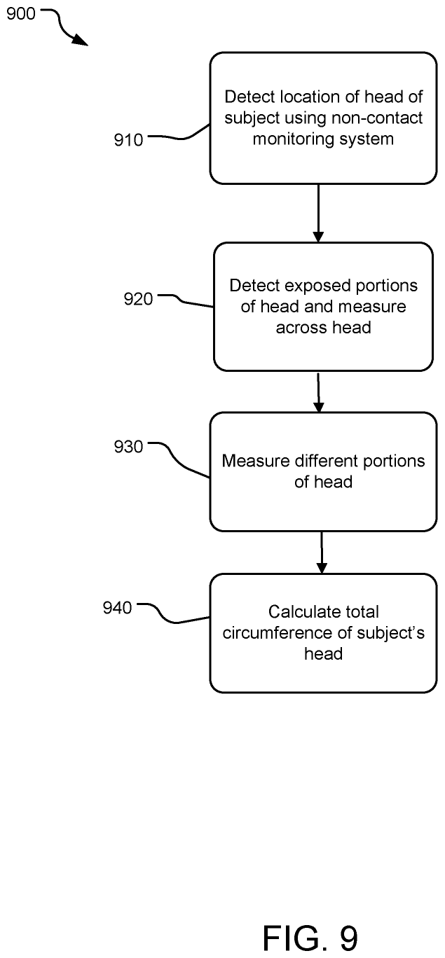

In other examples, the measured distance is only part of determining a desired physical or physiological characteristic, and further measurements and/or calculations are required before obtaining the desired physical or physiological characteristic. For example, and as described in further detail below with respect to FIG. 9, the desired physical or physiological characteristic may be head circumference. The non-contact monitoring system may be used to identify a first side of the subject's head and a second, opposite side of a subject's head, and then may be used to measure the distance between these two points. This distance provides a segment of the overall head circumference measurement. Further monitoring of the subject (which may be continuous or periodic) may identify instances when other parts of the subject's head are visible to the non-contact monitoring system (i.e., when the subject has rolled on to his or her side, thus exposing the side of the subject's head), at which point additional segments of the subject's head circumference can be measured using the same process as described previously. This process may be repeated until all portions of the subject's head have been measured, at which point a circumference can be calculated by adding the measured segments, being careful to eliminate any overlap between measurements. Alternatively, an estimate as to the subject's head circumference can be calculated using, e.g., one measured segment and a known correlation formula.

While the previous discussion has described instances of measuring from a subject's head to a subject's feet, or from one side of a subject's head to opposite side of a subject's head, it should be appreciated that the method described herein can involve measuring between any two features on a subject. Non-limiting examples include measuring a subject's abdomen, chest, or tibia.

FIG. 7 provides a method 700 for determining a length of a subject (e.g., infant, neonate, child) using a non-contact monitoring system as described herein. In a first step 710, the head of the subject is detected with the non-contact monitoring system utilizing depth; the depth may be determined from depth measurements, from reflected signals, or from reflected intensity signals, for light, IR, RGB, etc. AI may be utilized to detect and/or confirm the top of the head. In a second step 720, the non-contact monitoring system detects the foot of the subject utilizing depth; the depth may be determined from depth measurements, from reflected signals, or from reflected intensity signals, for light, IR, RGB, etc. Again, AI may be utilized to detect and/or confirm the feet or foot. It is noted that steps 710 and 720 may be done in reverse order (with the foot detected before the head) or may be done simultaneously. In a step 730, the non-contact monitoring system determines the distance between the head (detected in step 710) and the foot (detected in step 720) as a linear measurement, and in a step 740, this distance is outputted as the length of the subject.

If the subject is not arranged in a straight line, e.g., the infant is in a fetal position, the length of the subject may, nevertheless, be determined. For example, the length may be determined by finding the ridge, or highest line, extending from the head to the feet, and determining that distance as multiple segments (e.g., linear segments) between points such as the head, shoulder, hip, knee, etc. AI may be used to determine or confirm the position of the subject and identify the features (e.g., head, shoulder, hip, knee, etc.). In another example, the length may be determined by a search algorithm (e.g., A* search algorithm).

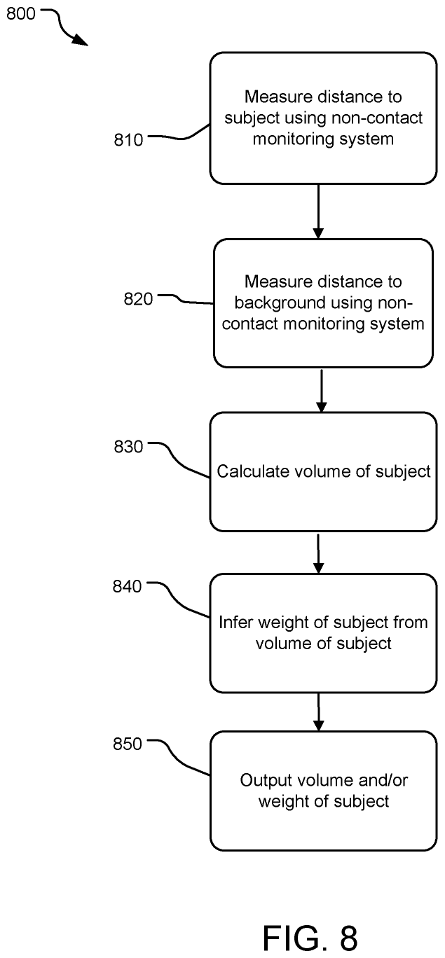

FIG. 8 provides a method 800 for determining a volume and thus the weight of the subject using a non-contact monitoring system as described herein. In a first step 810, (essentially) the entire exposed surface of the subject is detected with the non-contact monitoring system using depth signals, reflection signals, or light intensity signals, creating a topographical map. In a second step 820, which can be done prior to the first step 810, simultaneously with, or after the first step 810, the background (e.g., crib mattress) is detected. From the topography map of the subject created in the step 810 and the background created in the step 820, the volume of the subject is calculated in step 830. In step 840, from the volume of step 830, the weight of the subject is inferred using a previous mapping or function that relates the weight of the subject to the volume of the subject. In a step 850, this volume and/or weight is outputted from the system.

In an alternate method, the volume and/or weight of the subject can be determined by creating a topographical map of the subject from several positions, e.g., while the subject is on its front, on its side, on its back, etc., and those maps registered e.g., using point-set or point-cloud registration, to get a three-dimensional model of the subject.

From the volume and the weight, the body mass index (BMI) can be calculated.

Head circumference can be determined by the non-contact monitoring system by generating length measurements across the exposed surface of the skull/head, e.g., from edge to edge, and inferring a total circumference or by creating a three-dimensional mapping of the head.

FIG. 9 provides a method 900 for determining head circumference of the subject using a non-contact monitoring system as described herein. In a first step 910, the head of the subject is found, optionally with AI being used to detect and/or confirm the head. In a second step 920, at the appropriate location to measure circumference of the head, the length of the exposed head surface, from edge to edge, is measured with the non-contact monitoring system to obtain a first length; this measurement can be based on the shape of the subject, e.g., the shape of the head. Over time, as the subject changes position and different portions of the head are exposed, the length is obtained for each exposed surface segment in step 930, and in step 940, the total circumference of the head is calculated from the multiple measured segments.

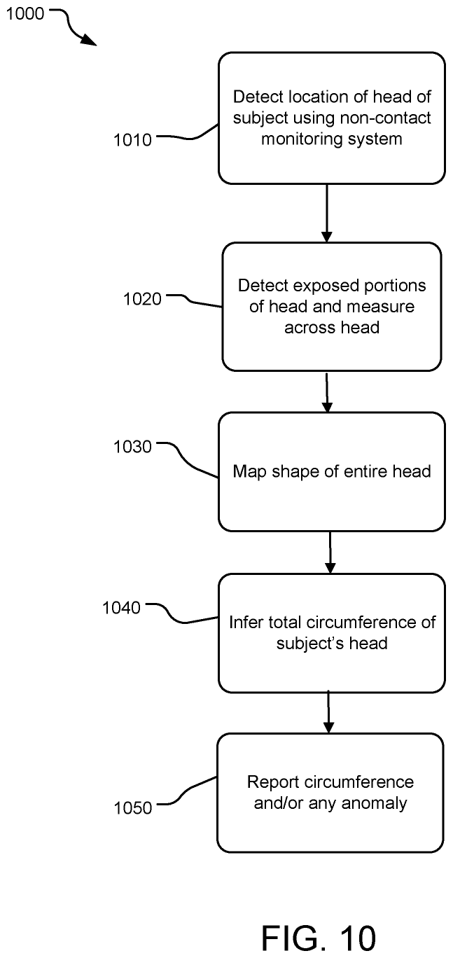

FIG. 10 provides another method 1000 for determining head circumference of the subject using a non-contact monitoring system as described herein. In a first step 1010, the head of the subject is found, optionally with AI being used to detect and/or confirm the head. In a second step 1020, (essentially) the entire exposed surface of the subject's head, at the appropriate location to measure circumference of the head, is detected with the non-contact monitoring system, determining a distance or length across the mapped exposed surface. Over time, as the subject rolls over from side to side, a complete morphology of the head is mapped in step 1030, e.g., using point-set or point-cloud registration, and in step 1040, the total circumference of the head is inferred from the map. Not only can the circumference of the head be monitored for typical subject growth, conditions such as plagiocephaly (flat head) and hydrocephalus can be detected and monitored by the map. Any anomaly can be reported to the clinician or caregiver in step 1050, as well as the circumference obtained from the mapped head.

Other topographical anomalies can be determined by the systems 100, 200 and variations thereof and reported to the clinician or caregiver. For example, an anomaly such as a curved spine can be discovered and monitored over time, in some instances, sooner than a visual diagnosis. Also, any irregular growth of an appendage or limb can be monitored over time.

The non-contact monitoring systems 100, 200 and variations thereof can be used to determine milestones such as smiling, lifting one's head, grasping objects, rolling over, and sitting up. As discussed in greater detail previously, the systems 100, 200 can be used to identify features on a subject. Identifying features on a subject can be carried out using depth measurements, using software such as facial recognition software, using artificial intelligence, or any combination thereof. In some embodiments, the feature or features identified by the systems are monitored over time, and the change in one or more distances between identified features is used to identify a developmental milestone. For example, the systems 100, 200 can first identify as features to monitor a subject's eyes and the corners of a subject's mouth. By measuring the change in distance between these features, developmental milestones such as smiling may be identified by the system. For example, a decrease in the distance between the eyes and corners of the mouth may indicate smiling. In another example, a three-dimensional map of the subject's mouth can be created as discussed previously (e.g., by taking multiple depth measurements of the subject's mouth. Tracking this three-dimensional map over time may allow for identification of smiling.

Another developmental milestone that can be monitored using the technology described herein includes the development of language, including, but not limited to, laughing, babbling, and talking. The system may utilize the microphone 140 described previously to detect audible noises produced by the subject. Audio signals received by microphone 140 may be processed and analyzed, including using artificial intelligence, to determine what type of language development is being detected (e.g., laughing, babbling, talking, etc.). The audio signals received by the microphone 140 may be processed in conjunction with monitoring the subject's mouth as discussed previously to aid in confirming the development of language skills and avoid false positives (e.g., when the sound picked up by the microphone 140 is not produced by the subject). For example, when a sound is detected by the microphone and analyzed to identify, e.g., laughing, babbling, talking, etc., the system may also consider the movement of the subject's mouth at the time the sound was detected. If the movement of the subject's mouth does not align with the language type determined by analyzing the detected sound, or if the subject's mouth is not moving at all, then this may denote a false positive.

With the depth measurements from the non-contact monitoring systems 100, 200, movement and motion, including specific motions, of the subject can be also be detected. The systems 100, 200 and variations thereof can be used to determine specific milestones of fine or gross motor skill development, such as lifting one's head, grasping objects, rolling over, sitting, etc. Reaction (e.g., movement) due to a stimulus, such as to a loud sound to determine level of hearing or to a bright light to determine level of vision, can be monitored. To monitor movement and motion, the systems 100, 200 can recognize a first position of the subject or a portion of the subject, recognize when movement to a second position has occurred, and recognize what that change in position represents. Recognition of the first position and the second position can be via depth measurements to one or more points on the subject's body as described previously, creating a three-dimensional map of the subject's body or portion of the subject's body as described previously (including using multiple depth measurements to create the three-dimensional map), and/or via use of artificial intelligence. Analysis of the change in position can be via comparison to known dimensional changes indicative of certain movements and/or via use of artificial intelligence.

In the example of monitoring for lifting of the subject's head, the system can identify as a feature the subject's head (including via use of artificial intelligence), measure the depth distance to one or more locations on the subject's head, and monitor the change in these depth measurements over time. When the depth measurements decrease, this may be indicative of the subject lifting its head. A similar analysis may be used to monitor sitting up, with a larger decrease in depth measurement to the subject's head being indicative of the subject sitting up in some cases. Rolling over may be monitored and identified in a variety of different ways, one of which may include identifying the subject's shoulder and monitoring the depth measurement to the subject's shoulder. When the subject is lying down on its back, the depth measurement to a shoulder is calculated. As the subject begins to roll over, the depth measurement to the shoulder decreases as it moves closer to the shoulder. If the shoulder depth measurement then returns to a value similar to the original shoulder measurement, but with the shoulder being identified in a location a distance away from the location of the shoulder in the original measurement (including, in some cases, the change in location of the shoulder being a distance at least equal to the width of the subject), this may be indicative of the subject rolling over.

The system described herein can use the occurrence of one or more detected developmental milestones to compare against standardized development timelines. In instances where one or more detected developmental milestones occur outside of a standardized development timeline, this can be used to suggest potential further treatment of the subject, such as physical or speech therapy. Table 1 shows an exemplary standardized developmental timeline that may be used as a point of comparison for developmental milestones detected by the non-contact monitoring system described herein.

TABLE 1

| Gross | | | | |
|---|---|---|---|---|
| Motor | Fine Motor | Language/Cognitive | Social | |
| 1 mo. | Moves head | Strong grip | Stares at hands and | Tracks movement |
| | from side to | | fingers | with eyes |
| | side when | | | |
| | on stomach | | | |
| 2 mo. | Holds head | Opens and closes | Begins to play with | Smiles responsively |
| | and neck up | hands | fingers | |
| | briefly while | | | |
| | on tummy | | | |
| 3 mo. | Reaches and | Grips objects in | Coos | Imitates you when |
| | grabs at | hands | | you stick out |
| | objects | | | your tongue |
| 4 mo. | Pushes up on | Grabs objects -- and | Laughs out loud | Enjoys play and may |
| | arms when | gets them! | | cry when playing |
| | lying on | | | stops |
| | tummy | | | |
| 5 mo. | Begins to roll | Is learning to transfer | Blows “raspberries” | Reaches for mommy |
| | over in one or | objects from one | (spit bubbles) | or daddy and cries if |
| | the other | hand to the other | | they're out of sight |
| | direction | | | |
| 6 mo. | Rolls over | Uses hands to “rake” | Babbles | Recognizes familiar |
| | both ways | small objects | | faces --caregivers |
| | | | | and friends as well |
| | | | | as family |
| 7 mo. | Moves | Is learning to use | Babbles in a more | Responds to other |
| | around --is | thumb and fingers | complex way | people's expressions |
| | starting to | | | of emotion |
| | crawl, scoot, | | | |
| | or “army | | | |
| | crawl” | | | |
| 8 mo. | Sits well | Begins to clap hands | Responds to familiar | Plays interactive |
| | without | | words, looks when | games like peekaboo |
| | support | | you say their name | |
| 9 mo. | May try to | Uses the pincer grasp | Learns object | Is at the height of |
| | climb/crawl | | permanence -- that | stranger anxiety |
| | up stairs | | something exists | |
| | | | even if they can't see | |
| | | | it | |
| 10 mo. | Pulls up to | Stacks and sorts toys | Waves bye-bye | Learns to understand |
| | stand | | and/or lifts up arms | cause and effect (“I |
| | | | to communicate “up” | cry, Mommy |
| | | | | comes”) |
| 11 mo. | Cruises, | Turns pages while | Says “mama” or | Uses mealtime |
| | using | you read | “dada” for either | games (dropping |
| | furniture | | parent | spoon, pushing food |
| | | | | away) to test your |
| | | | | reaction; expresses |
| | | | | food preferences |
| 12 mo. | Stands | Helps while getting | Says an average of 2- | Plays imitative |
| | unaided and | dressed (pushes | 3 words (often | games such as |
| | may take first | hands into sleeves) | “mama” and “dada”) | pretending to use the |
| | steps | | | phone |

The results from any of the physiological measurements, determined characteristics, or monitored parameters may be shown on a screen such as the display 122, 222 of the non-contact monitoring system 100, 200, respectively, or may be transmitted to the remote device 130, 230 for viewing by the clinician or caregiver. Additionally or alternately, the data may be automatically transmitted to the subject's patient record or file. Over time (e.g., days, weeks, months, etc.), a report can be generated showing any change in the measurements. These may be compared to one another and to standardized growth curves. If the subject's growth or progress is outside of the predetermined “normal” range or if a drastic change is observed, the system 100, 200 may alert the clinician or caregiver.

As discussed previously, the systems and methods described herein may rely on artificial intelligence for one or more aspects of the disclosed technology. In some embodiments, artificial intelligence is used in identifying a body part or body region of the subject (e.g., the subject's head, the subject's foot, etc.) and/or to identify features of the subject within or on the subject's body part or body region. In some embodiments, artificial intelligence is used to carry out the correlations or estimates discussed herein with respect to determining physical or physiological characteristics of a subject or determining the occurrence of developmental milestones. In some embodiments, depth measurements obtained from the non-contact monitoring system are used by the artificial intelligence to calculate physical or physiological characteristics of a subject or to determine the occurrence of developmental milestones.

The computing device of the systems described herein can be trained with vision-based artificial intelligence (AI) methods to learn to identify these body parts, regions, and/or features. The computing device of the systems described herein can be trained using any standard AI model and standard methods, e.g., utilizing numerous data points to create a dataset of images.

The above specification and examples provide a complete description of the structure and use of exemplary embodiments of the invention. The above description provides specific embodiments. It is to be understood that other embodiments are contemplated and may be made without departing from the scope or spirit of the present disclosure. The above detailed description, therefore, is not to be taken in a limiting sense. For example, elements or features of one example, embodiment or implementation may be applied to any other example, embodiment or implementation described herein to the extent such contents do not conflict. While the present disclosure is not so limited, an appreciation of various aspects of the disclosure will be gained through a discussion of the examples provided.

Unless otherwise indicated, all numbers expressing feature sizes, amounts, and physical properties are to be understood as being modified by the term “about,” whether or not the term “about” is immediately present. Accordingly, unless indicated to the contrary, the numerical parameters set forth are approximations that can vary depending upon the desired properties sought to be obtained by those skilled in the art utilizing the teachings disclosed herein.

As used herein, the singular forms “a”, “an”, and “the” encompass implementations having plural referents, unless the content clearly dictates otherwise. As used in this specification and the appended claims, the term “or” is generally employed in its sense including “and/or” unless the content clearly dictates otherwise.

1. A method of monitoring a subject, the method comprising:
detecting a first feature and a second feature of the subject in a field of view of a non-contact monitoring system;
determining, with the non-contact monitoring system, a measurement between the first feature and the second feature; and
correlating the measurement to a measurement of a physiological characteristic of the subject.

2. The method of claim 1, wherein detecting the first feature and the second feature of the subject in a field of view of a non-contact monitoring system comprises using depth measurements obtained by the non-contact monitoring system to detect the first feature of the subject, the second feature of the subject, or both.

3. The method of claim 1, wherein detecting the first feature and the second feature of the subject in a field of view of a non-contact monitoring system comprises using artificial intelligence to detect the first feature of the subject, the second feature of the subject, or both.

4. The method of claim 1, wherein the measurement between the first feature and the second feature is a linear measurement.

5. The method of claim 1, wherein the first feature is the top of the head of the subject, the second feature is a foot of the subject, and the physiological characteristic of the subject is the height of the subject.

6. The method of claim 1, wherein the measurement between the first feature and the second feature comprises two or more linear segment measurements.

7. The method of claim 1, further comprising:
prior to detecting the first feature and the second feature of the subject in a field of view of a non-contact monitoring system, detecting a body part or region of the subject, wherein the first feature and the second feature are located on or within the detected body part or region.

8. The method of claim 7, wherein the body part or region is the subject's head.

9. The method of claim 1, wherein detecting the first feature and the second feature of the subject in a field of view of a non-contact monitoring system comprises creating a three-dimensional map of a region or body part of the subject from depth measurements obtained by the non-contact monitoring system, wherein the first feature and the second feature are located on or within the body part or region.

10. A method of monitoring a subject, the method comprising:
detecting at least a first feature of the subject in a field of view of a non-contact monitoring system;
determining, with the non-contact monitoring system, a first depth measurement to the at least first feature at a first time;
determining, with the non-contact monitoring system, a second depth measurement to the at least first feature at a second time;
comparing the first depth measurement to the second depth measurement; and
from the comparison of the first depth measurement to the second depth measurement, determining whether the subject has performed a specific movement or action.

11. The method of claim 10, wherein the specific movement or action is selected from the group consisting of smiling, lifting the head, rolling over, sitting up, or grasping an object.

12. The method of claim 10, wherein detecting the first feature of the subject in the field of view of the non-contact monitoring system comprises using one or more depth measurements obtained by the non-contact monitoring system to detect the first feature of the subject.

13. The method of claim 10, wherein detecting the first feature of the subject in the field of view of the non-contact monitoring system comprises using artificial intelligence to detect the first feature of the subject.

14. The method of claim 10, wherein determining whether the subject has performed a specific movement or action comprises using artificial intelligence to determine the movement or action performed by the subject.

15. The method of claim 10, further comprising:
prior to detecting at least the first feature of the subject in a field of view of the non-contact monitoring system, detecting a body part or region of the subject, wherein the first feature is located on or within the detected body part or region.

16. The method of claim 15, wherein detecting a body part or region of the subject comprises using artificial intelligence to detect the body part or region.
